(19) United States
(12) Patent Application Publication
(10) Pub. No.: US 2025/0380854 A1
(43) Pub. Date: Dec. 18, 2025

(54) GUIDED CONNECTION FOR ENDOSCOPE REPROCESSING

(71) Applicant: Medivators Inc., Minneapolis, MN (US)

(72) Inventors: Fabio Nazzarri, Rome (IT); Massimo RUFFINO, Pomezia (RM) (IT)

(21) Appl. No.: 18/876,913

(22) PCT Filed: May 4, 2023
(86) PCT No.: PCT/US2023/066603

§ 371 Date: Dec. 19, 2024

Related U.S. Application Data
(60) Provisional application No. 63/367,980, filed on Jul. 8, 2022.

(51) Int. Cl. A61B 1/00; A61B 1/12

(52) U.S. Cl. CPC A61B 1/00128; A61B 1/125

(57) ABSTRACT

Various embodiments disclosed relate to a connector for use in an endoscope reprocessing system. The present disclosure includes a universal connector having three blocks. The first block can be configured for aligning a receptacle to the reprocessing system in a first direction. The second block can be connectable to the first block and configured for aligning the receptacle to the reprocessing system in a second direction. The third block can be configured to receive one or more hook-ups.

### CLAIM OF PRIORITY

This patent application claims the benefit of priority to U.S. Provisional Patent Application Ser. No. 63/367,980, filed Jul. 8, 2022, which is incorporated by reference herein in its entirety.

### BACKGROUND

Endoscopy includes medical procedures leveraging endoscopes to look inside the body. For example, an endoscope can be used to examine the interior of a body cavity or hollow organ. Endoscopes can be used, for example, in investigating digestive symptoms such as gastrointestinal bleeding, vomiting, abdominal pain, nausea, or others. For such procedures, an endoscope can be directly inserted into an orifice or organ.

A variety of types of endoscopes exist, many of which are designed to be reusable due to their complex or precise components. Endoscopes can include, for example, rigid or flexible tubing. In some cases, a light source and camera optics can be used in conjunction with a light delivery system for imaging and visualization during the endoscopy. Such a light delivery system can include lenses, camera sensors, fibers, or other appropriate components for imaging. Some endoscopes can additionally include separate channels for use of other medical instruments therein.

Due to the nature of endoscopy, reusable endoscopes must be high level disinfected, according to national guidelines or other medical standards of care. High level disinfection of flexible endoscopes is commonly referred to as reprocessing. Such reprocessing can include a multi-step process including pre-cleaning, leak testing, manual cleaning, cleaning verification, visual inspection, high level disinfection, rinsing, drying, and storage. Without correct reprocessing, residual contamination can remain on endoscopes.

In endoscope reprocessing systems, such as automated endoscope reprocessors (AERs), one or more endoscopes in need of reprocessing are coupled to the system through a hook-up specific to that type of endoscope. The endoscope is then washed, disinfected, and dried. The hook-ups to the specific endoscopes being reprocessed should be secure connections.

### SUMMARY OF THE DISCLOSURE

The present disclosure provides a connector device and methods for use with an endoscope reprocessing system, such as to reduce operator touch and movement when connecting an endoscope to the reprocessing system. This can help increase the efficiency of cleaning endoscopes within the disinfection process.

In endoscope reprocessing systems, one or more endoscopes can be hooked up to a cleaning and disinfecting flushing line for disinfection of various channels in the endoscopes. Typically, different types of endoscopes (e.g., colonoscopes versus esophagoscopes) or different brands of endoscopes provide different types of hook-up connectors for cleaning and reprocessing of these channels. When connected, these hook-ups should be secure and provide a fluid-tight connection to the endoscope in order to fully achieve disinfection results.

The endoscope reprocessing connector discussed herein can be used as a universal connector, to provide interchangeable connectivity for multiple types or forms of endoscope hook-ups. This endoscope reprocessing connector can include three blocks. The first block can be fixed on a lateral side of a cleaning chamber, such as to allow for vertical insertion and removal of a plug-in receptacle and endoscope hook-up. The second block can be fixed on a lateral top side of the receptacle to allow sliding movement of the receptacle within the reprocessing machine and lateral alignment of the hook-up. The third block can be on a lateral bottom side of the receptacle, such as to create a coupling between the removable hookup for the endoscope and the devices chosen for use. In some cases, the device can include an automated release allowed by an automated actuator that pushes in the bottom block through a lateral side of the device.

The presently described endoscope reprocessing connector can be advantageous by allowing for simpler movements when securing the endoscope to the reprocessor, while providing increased ease of use for operators, and the use of less expensive. This results in reduced touch and alteration by the operator, and increased accuracy.

For instance, with the presently described connector, the operator can, while outside of the reprocessing system, couple endoscope connectors to a specific endoscope hook-up, plug the hook-up to a receptacle, and lift the receptable by lateral handles to move the receptacle within the reprocessing system. The operator can, while outside of the reprocessing system, activate opening a chamber in the reprocessing system and place the receptacle from a top side of the reprocessing system, following a vertical guide therein, to place the receptacle down into the opened chamber. In such a system, the operator can move a single handle (or induce an automated actuator) to couple the receptacle with connections within the chamber. With these movements, the connection can be completed with the receptacle in position for reprocessing, chiefly outside the system, with a total reduced operator touching of equipment compared to alternative reprocessing systems.

Additionally, the disclosed reprocessing system with universal connector can be easier to use than other reprocessing systems. Specifically, the endoscope being reprocessed can have its connectors hooked up while staying external to the reprocessing machine, yet subsequently the operator can connect the receptacle to the machine while avoiding touching internal surfaces of the reprocessing machine. This can also help increase safety of endoscope reprocessing and reduce opportunities for cross-contamination.

In an example, a universal connector assembly for securing a receptacle to a reprocessing system can include a first block configured for aligning the receptacle to the reprocessing system in a first direction, a second block connectable to the first block, the second block configured for aligning the receptacle to the reprocessing system in a second direction, and a third block including one or more hook-ups.

In an example, a reprocessing system can include a cleaning chamber, a tray separable from the system, the tray for insertion into the cleaning chamber, and a universal connector configured to secure the tray to the reprocessing system in the cleaning chamber. The universal connector can include a first block configured for vertical connection of the tray to the reprocessing system, a second block connectable to the first block, the second block configured for lateral connection of the tray to the reprocessing system, a third block comprising one or more endoscope hook-ups, and a connecting portion actuatable for securing the second block to the third block and connecting the tray to the reprocessing system.

In an example, a method of connecting an endoscope to an endoscope reprocessing system can include laterally inserting a receptacle into a cleaning chamber of the reprocessing system and vertically aligning the receptacle to a first portion of a connector, vertically aligning the receptacle within the cleaning chamber with a second portion of the connector and causing one or more endoscope connectors to be hooked up to the receptacle with a third portion of the connector.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIGS. 1A-1D depict perspective views of an endoscope reprocessing system using a universal connector in an example.

FIGS. 2A-2D depict perspective views of a tray for an endoscope reprocessing system using a universal connector in an example.

FIGS. 3A-3B depict perspective views of a universal connector for an endoscope reprocessing system in an example.

FIGS. 4A-4B depict insertion of a tray into an endoscope reprocessing system with a universal connector in an example.

FIGS. 5A-5B depict manual connection of a tray into an endoscope reprocessing system with a universal connector in an example.

FIGS. 6A-6B depict automated connection of a tray into an endoscope reprocessing system with a universal connector in an example.

FIGS. 7A-7B depict connection of a tray into an endoscope reprocessing system with a universal connector in an example.

FIG. 8 depicts a flow chart describing a method of connecting a tray to an endoscope reprocessing system in an example.

### DETAILED DESCRIPTION

The present disclosure describes, among other things, a universal connector for an endoscope cleaning process and system. The universal connector can be used with a variety of hook-ups for endoscope reprocessing systems. The universal connector reduces the amount of time needed to situate a new container therein. In some cases, a coupling of the universal connector can be automated. Benefits of the universal connector include less touch movements, easy to use, and use of potentially cheaper arrangements than previous connectors. In some cases, the universal connector can be part of a receptacle, tray, rack, or container used in the reprocessing system.

The universal connector can include three blocks: a first block for vertical insertion and removal of a container and hook up, such as in a configuration where this first block can be on a lateral side of the cleaning chamber and allow for alignment in a first direction; a second block on a top side of the container that connects to the first block through a set of linking handle arms which allow for alignment in a section direction; and a third block on a bottom side of the container that creates coupling between the removeable hook-up and the devices chosen for use.

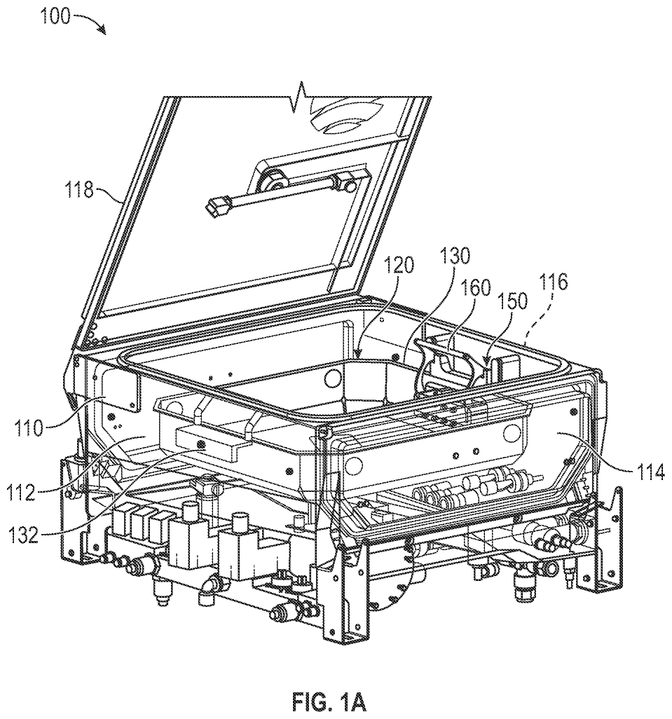

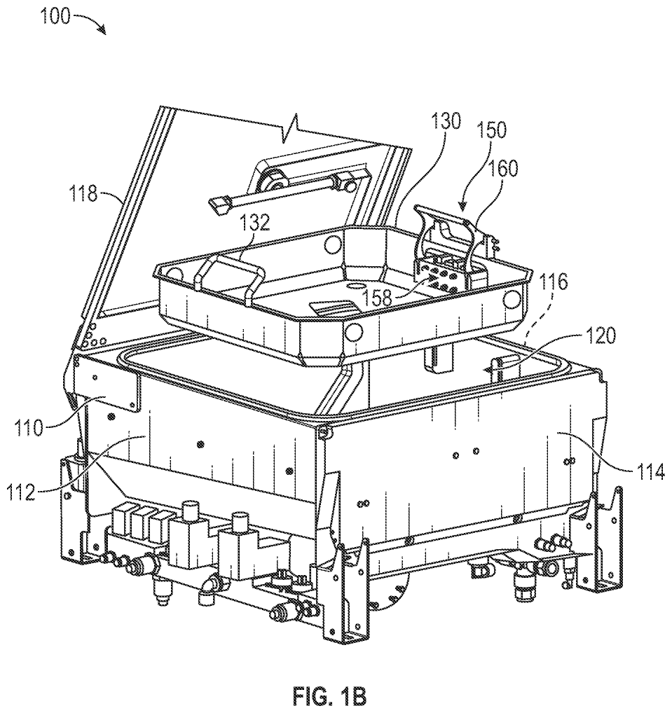

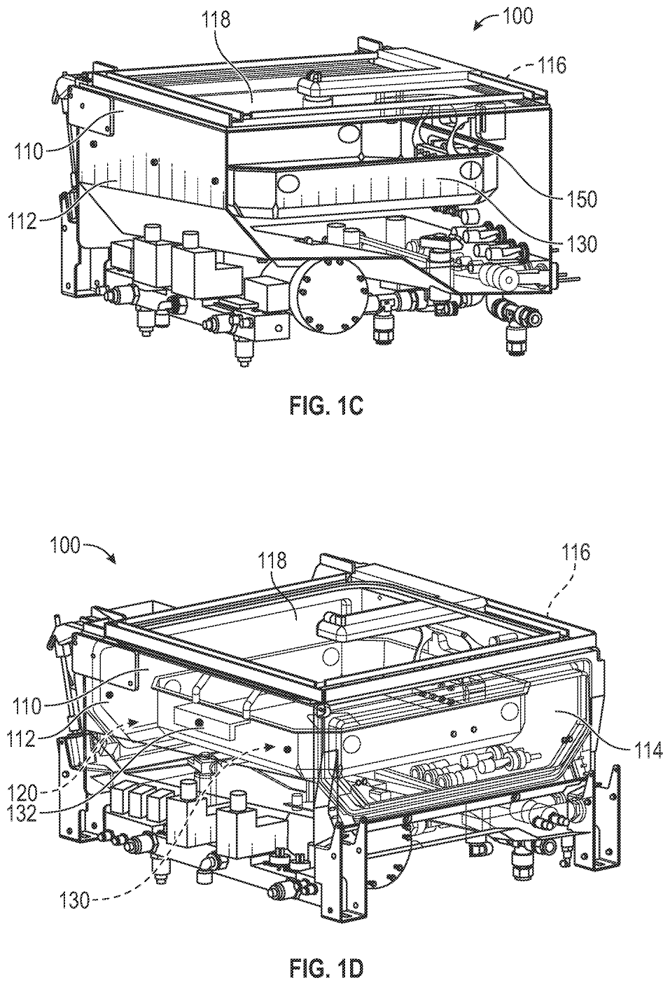

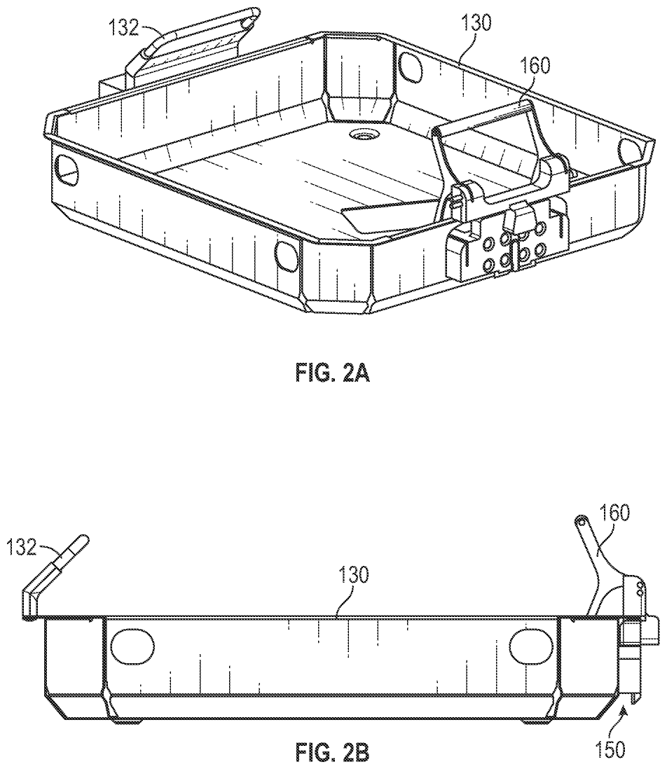

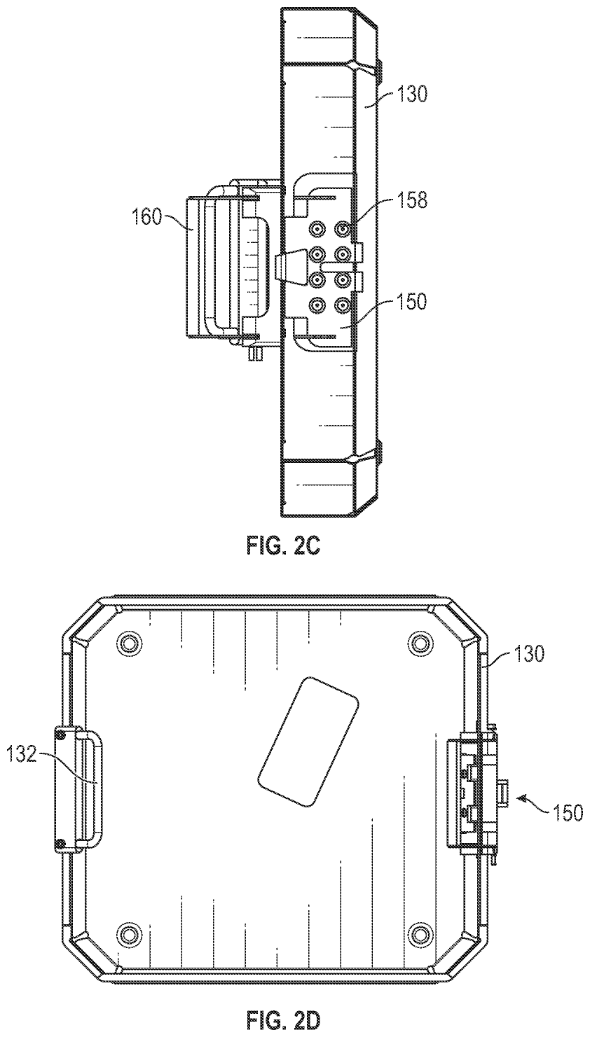

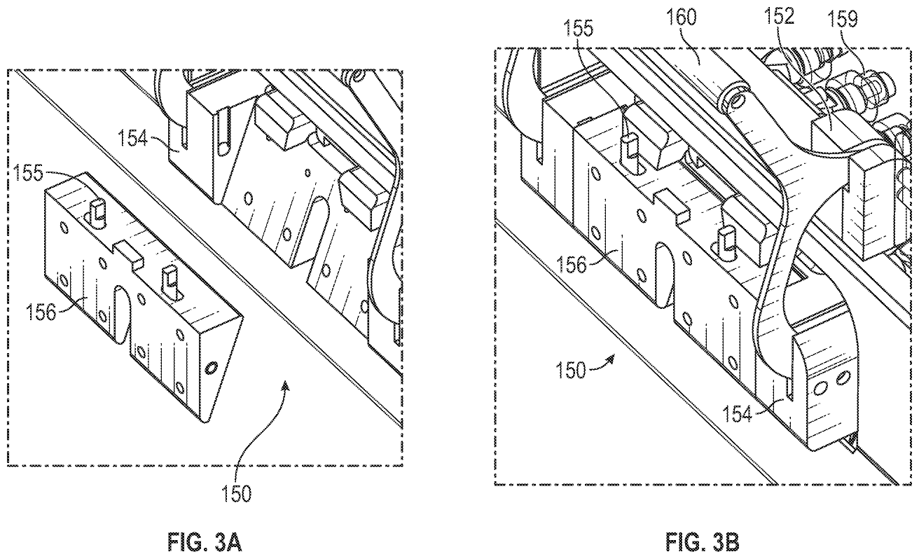

FIGS. 1A-1D depict perspective views of an endoscope reprocessing system 100 using a universal connector in an example, while FIGS. 2A-2D and 3A-3B depicts closer views of components of the system 100. The system 100 can include a housing 110 with sides 112, 114, 116, and top 118, and chamber 120, a receptacle 130 having handles 132 and connector 150 with pins 136, the connector 150 having a first block 152, a second block 154 with an attachment mechanism 155, a third block 156 with endoscope hook-ups 158, and an actuator 160 extending between the first block 152 and the second block 154.

The endoscope reprocessing system 100 can be used for processing or reprocessing medical devices with minimal operator touch to produce sterilized, decontaminated, disinfected, or clean devices. Reprocessing can include cleaning, disinfecting, decontaminating, sterilizing, or combinations thereof, medical devices used in an earlier procedure, such as endoscopes used in an endoscopic procedure. Reprocessing can include liquid and gaseous forms of reprocessing, such as with a detergent, a liquid sterilant, a steam sterilant, a steam-gas mixture sterilant, a gas, or combinations thereof. These types of fluids can be used for rinsing, heat-up, cool-down, or combinations thereof, depending on the specific reprocess performed.

The endoscope reprocessing system 100 can be used for cleaning, disinfecting, and sterilizing flexible endoscopic devices, but may also be used for reprocessing of other medical instruments, such as other medical instruments containing one or more lumens. In the endoscope reprocessing system 100, the housing 110 can include the sides 112, 114, 116 and top 118, defining the enclosure. Within the enclosure can be the chamber 120, in which the receptacle 130 can be inserted with the handles 132. The receptacle 130 can include one or multiple endoscopes. The universal connector 150 can be situated on the receptacle 130, and can contain the first block 152, the second block 154, and the third block 156. The first block 152 and the second block 154 can be attached through the actuator 160. The third block 156 can be connectable to the second block 154.

The housing 110 can serve as an enclosure to contain and protect the other components of the system therein. The housing 110 can be defined, for example, by the sides 112, 114, 116, and 118. The chamber 120 can be within the housing 110 and can be sized and shaped for receipt of a receptacle, such as a tray or dish for holding one or more endoscopes during reprocessing. In the example system 100, the top 118 of the housing 110 can be openable for inserting and removal of such a receptacle.

Also seen in detail in FIGS. 2A-2D, a receptacle 130, can be a tray, basin, rack, or other object suitable for holding or hosting an endoscope within the system 100 for reprocessing. In some cases, the receptacle can be fitted with a connection block to secure the receptacle 130 within the chamber 120. In some cases, the receptacle can include two handles 132 for easy removal and insertion of the receptacle 130 instead of an operator touching inside the chamber 120 and the receptacle 130. The handles 132 can be gripped and lifted up.

The receptacle 130 can be sized and shaped such that an endoscope can be placed therein, such as in a folded state. Once inserted into the receptacle 130, the endoscope can be hooked up through the universal connector 150 to various ports and hook-ups as desired. For example, flexible tubing can be used to fluidly connect the endoscope within the receptacle 130. The endoscope can have one or more lumens (e.g., channels) that are connected to the receptacle. The lumens of the endoscope, when properly attached to the reprocessing system, can be fluid-tight, to prevent leakages. The endoscope can have a variable number of lumens, such as a single lumen (e.g., for bronchoscopes), or seven lumens (e.g., for colonscopes). Such lumens can include, for example, channels such as a suction channel, an air channel, a jet channel, a water channel, a biopsy channel, an extra channel, an elevator (lift) channel, or combinations thereof. When the endoscope is connected, a leak test can be performed by flushing air into the system to create a pressure gradient and determine if/where leaks are present. This can help ensure that the correct fluid(s) are flushed through each channel and each channel, that the appropriate flow and pressure monitors are in place.

As used herein, a “hook-up” refers to a fluid connection between an endoscope and the reprocessing machine or receptacle, such as by using the universal connector 150. One or more “hook-ups” can be internal, such as in or on the third block of the universal connector. The hook-ups can fluidly connect one or more endoscopes held within the receptacle 130. The hook-ups can allow for fluid engagement and alignment with channels in the endoscope, such that fluid can be flushed through the channels, to disinfect the channels themselves. This can include suction channels, air channels, water channels, or other methods of flushing.

The receptacle 130 can additionally include a connector 150, which can work in conjunction with the universal connector 150 to allow for hook-up of an endoscope in the receptacle 130. In some cases, pins 136 can be situated in the connector 150 to allow for alignment and connection of the receptacle 130 with the universal connector 150.

Seen in detail in FIGS. 3A-3B, the connector 150 can be a three-block, universal connector configurable for ease of use with a various of endoscopes and endoscope connection port types. The universal connector 150 can include first block 152, second block 154, and third block 156, which are each different components that together form the universal connector 150.

The first block 152 can be for aligning the receptacle 130 to the reprocessing system 100 in a first direction. Shown in assembly 100, the first block 152 can be for initial alignment of the receptacle 130, such as vertical alignment. In some cases, the first block 152 can be for lateral alignment of the receptacle 130 within the system 100.

The first block 152 can be a fixed structure in the receptacle 130. The first block 152 can be sized and shape to receive one or more attachment mechanisms to the receptacle 130 connector 150, such as the pins 136.

The second block 154, by comparison, can be for aligning the receptacle 130 within the endoscope reprocessing system 100 in a second direction. For example, if the first block 152 is used to align the receptacle 130 in a vertical direction, the second block 154 can be used to further align the receptacle 130 in a lateral direction within the chamber 120.

The second block 154 can include an attachment mechanism 155, such as to secure the receptacle 130 to the second block 154. In some cases, the attachment mechanism 155 can be a spring-loaded attachment mechanism for releasable connection of the third block 156 to the second block 154.

The third block 156 can connect with the second block 154 and can contain one or more hook-ups 158. The hook-ups 158 can be, for example, fluid flushing ports, or other plugs unique for the type of the endoscope to be reprocessed. The third block 156 can be connected and detached from the second block 154, such as by levers, pins, or other mechanisms. In some cases, the attachment mechanism 155 can be a spring-loaded attachment piece. This can allow for easy attachment and detachment from the universal connector 150.

The third block 156 can be removable from the universal connector 150. For example, an operator can remove and replace the third block 156 with an alternative third block 156, such as to replace the types of endoscope hook-ups being used. In this way, a large variety of different types of endoscope hook-ups can be easily switched in and out into the universal connector 150.

The actuator 160 can be a movement mechanism that is actuatable for connecting the third block 156 to the first block 152 and the second block 154. For example, the actuator 160 can be a manual handle that can be pushed or pulled to secure the universal connector 150. In other examples cases, the actuator 160 can be an automated actuator, such as a pulling actuator induced by an electromechanical system (e.g., controlled by a controller circuitry).

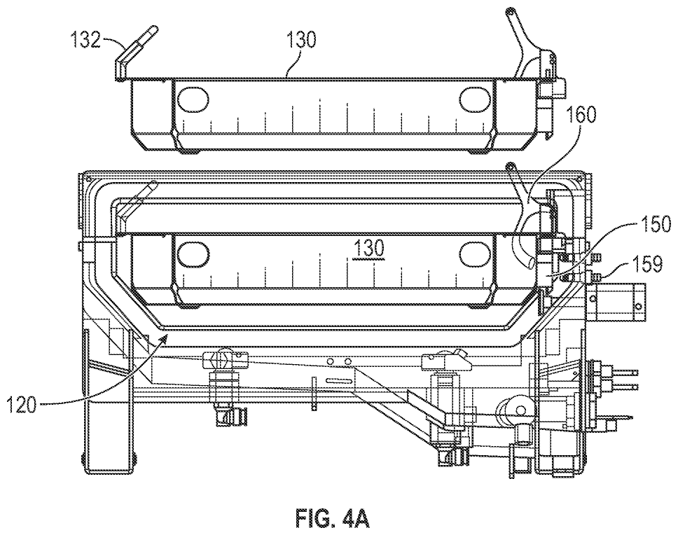

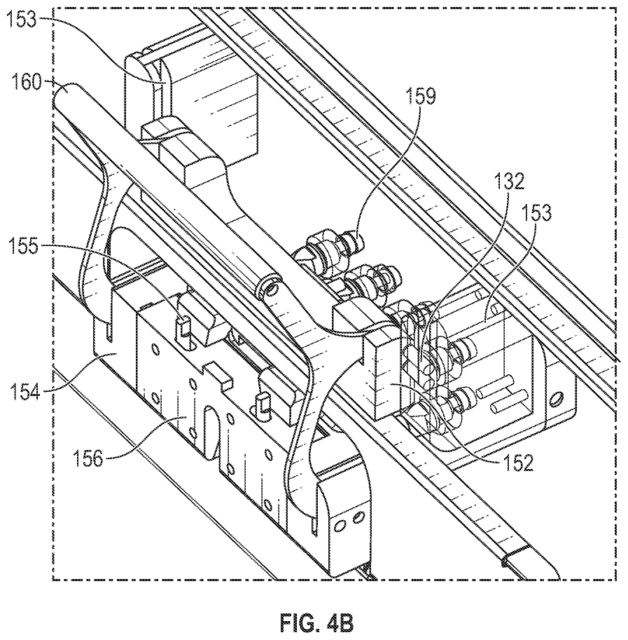

FIGS. 4A to 6B depict examples of securing an endoscope and a receptacle with a universal connector in an endoscope reprocessing system such as the system 100.

FIGS. 4A-4B depict a first step of inserting the receptacle 130 into the endoscope reprocessing system 100 with the first block 152 of the universal connector 150 in an example. In FIG. 4A, the receptacle 130 is placed in the chamber 120 by the handles 132. At this time, the endoscope can be within the receptacle 130 and appropriately hooked into the receptacle 130, such as by flexible tubing. The endoscope can be folded within the receptacle 130.

The handles 132 can allow an operator to reduce the amount of touching, contamination, and human contact in the chamber 120. As the receptacle 130 is lowered into the chamber 120, the first block 152 can allow for vertical alignment of the receptacle 130. For example, the first block 152 can include one or more slots or openings configured to align with pins or other receptors on the endoscope reprocessing system 100. Shown in FIG. 4B is vertical alignment of the pins 136 with slots 153 the system 100. Here, the first block 152 can help align and secure the receptacle 130 within the chamber 120 in a first direction.

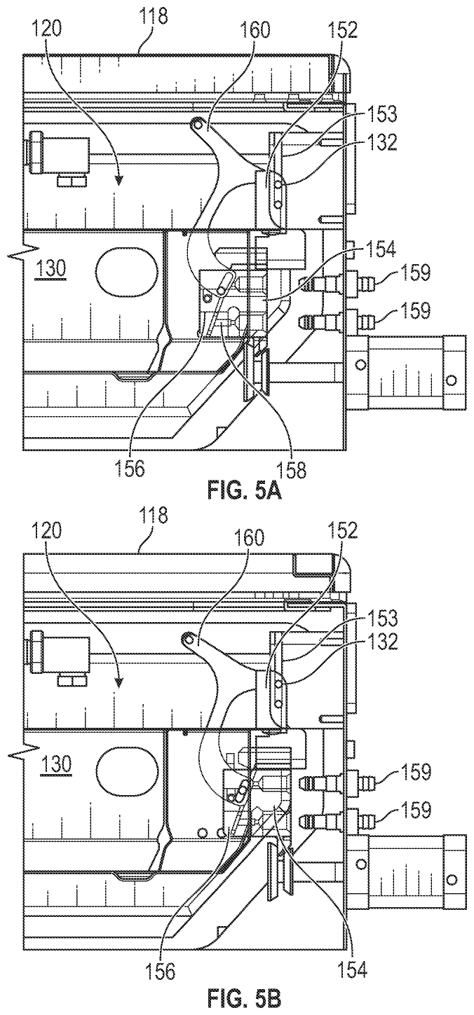

FIGS. 5A-5B depict a second step of inserting the receptacle 130 into the endoscope reprocessing system 100 with the second block 154 and the third block 156 of the universal connector 150 with a manual actuator in an example. In FIG. 5A, the receptacle 130 is already vertically aligned with the help of the first block 152.

The second block 154 can be attached to the first block 152 through an actuator 160, such as a handle. The third block 156 can be secured to the second block 154, such as through the attachment mechanism 155. In FIG. 5B, the third block 156 has been attached to the second block 154 and the actuator is induced to connect the universal connector 150 to the external fluid ports 159. The third block 156 can include one or more endoscope hook-ups 158. In some cases, the endoscope hook-ups 158 are not external.

The actuator 160 situated between the first block 152 and the second block 154 can allow for lateral movement of the second block 154 into the endoscope reprocessing system 100 sides 116 and fluid connection of the universal connector 150 to external fluid ports 159. For example, after the third block 156 is secured to the second block 154, the combined second block 154 and third block 156 can be laterally moved to align the receptacle 130. In this way, the endoscope hook-ups 158 can be fluidly connected to the external fluid ports 159 from the third block 156 through the second block 154 and through a side 116 wall of the endoscope reprocessing system 100 to the external fluid ports 159, allowing fluid flow through the external fluid ports 159 to the endoscope hooked into the endoscope hook-ups 158 on the inside of the third block 156. In this way, the endoscope can be situated inside the receptacle 130 and hooked up without an operator touching the inside of the chamber 120 or the receptacle 130.

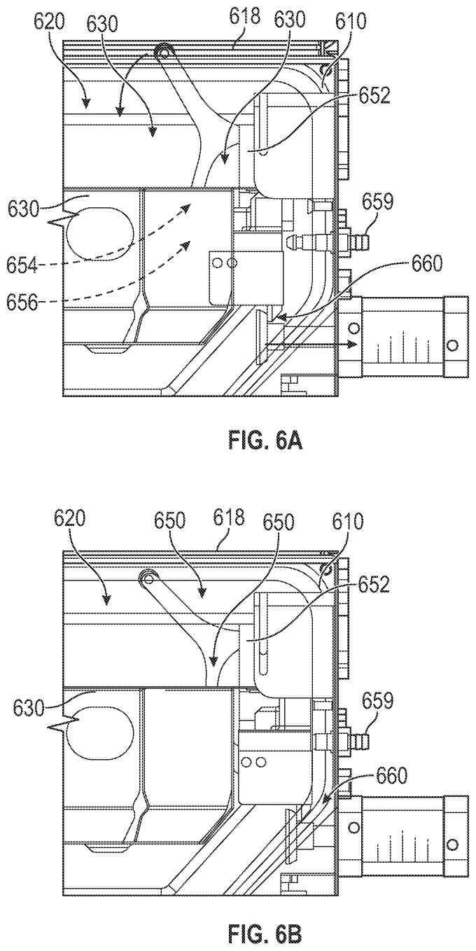

FIGS. 6A-6B depict steps of inserting a receptacle 630 into an endoscope reprocessing system 600 with a second block 654 and a third block 656 of the universal connector 650 with an automated actuator 660 in an example. The system 600 can include housing 610, top 618, chamber 620, receptacle 630, universal connector 650 with first block 652, second block 654, and third block 656, and actuator 660. The components of the system 600 can be similar to those, and connected in a similar fashion, of system 100 discussed above, except where otherwise noted.

In the example of FIGS. 6A-6B, instead of a manual handle for lateral movement of the universal connector 650, an automated actuator 660 is used. In this case, the system endoscope reprocessing system 600 can be in communication with an electromechanical actuation system operated by controller circuitry, such as to allow timed or trigger actuation of this step.

In an example, the controller circuitry can include a processor and a memory. The controller circuitry can include a timer or clock, and can be coupled to other actuators, sensors, and subsystems of the endoscope reprocessing system 600 or other related systems. The processor can be configured to implement functionality or process instructions for execution within the system 600, such as instructions stored in a program memory. The processor can execute the program instructions to cause the actuate the actuator 660 according to a specified schedule, threshold, or operator indication. The specified instructions can be retrieved from a data memory, for example. The processor can send one or more commands to the system 600. The processor also can also send or receive information from a user interface, generate logging or status information, and connect with other information systems (e.g., to record the time and state of when a receptacle was connected to the system 600).

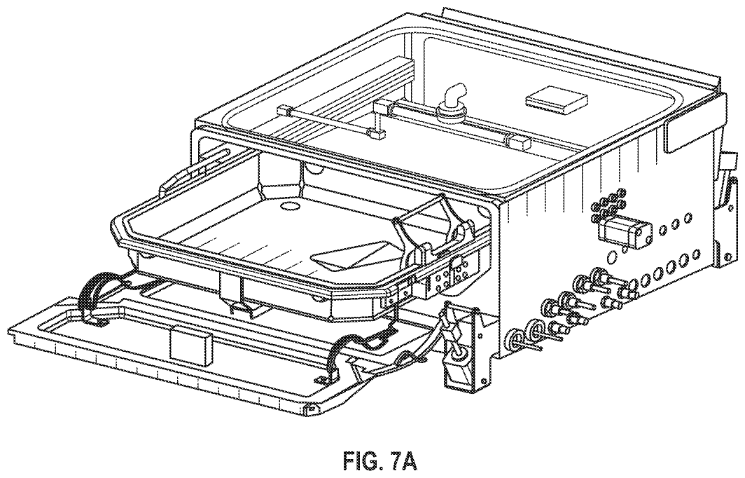

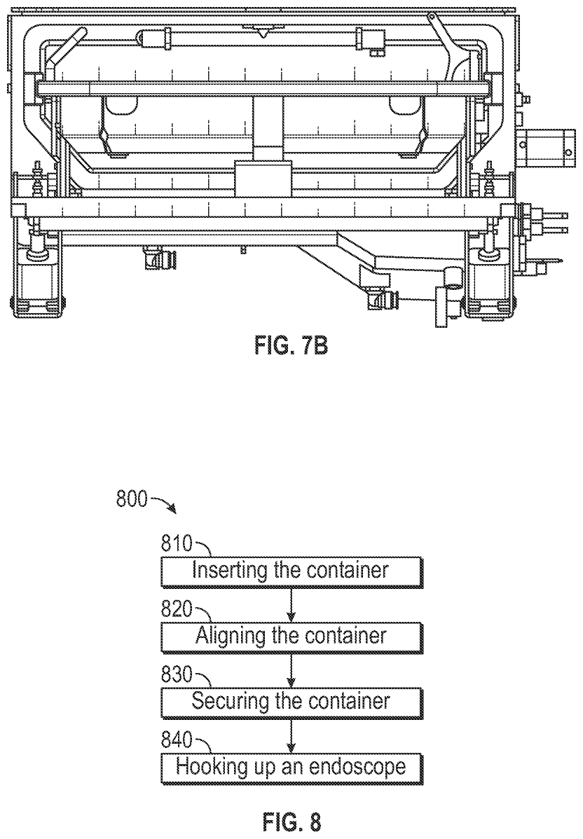

FIGS. 7A-7B depict connection of a receptacle into an endoscope reprocessing system 700 with a universal connector 750, wherein the receptacle is inserted laterally. The endoscope reprocessing system 700 can include housing 710, top 718, chamber 720, receptacle 730, universal connector 750 with first block 752, second block 754, and third block 756, and actuator 760. The components of the system 700 can be similar to those, and connected in a similar fashion, of system 100 discussed above, except where otherwise noted.

In the endoscope reprocessing system 700, the first block 752 can be configured for an initial lateral alignment of the universal connector 750, instead of the vertical alignment discussed above. In this case, the second block 754 can be used with the actuator 760 for a vertical alignment.

FIG. 8 depicts a flow chart describing a method 800 of connecting a receptacle to an endoscope reprocessing system in an example. The method 800 can be done, in some cases, by an operator. The method 800 can be automated in some cases, and controlled by controller circuitry, a computer, a robot, or an autonomous or semi-autonomous agent. The method 800 can include blocks 810 to 840. At block 810, a receptacle, such as a container with an endoscope, is inserted into an endoscope reprocessing machine. At block 820, the receptacle is aligned in a first direction with a first block of a universal connector. At block 830, the receptacle is aligned in a second direction with a second block of a universal connector and secured.

Finally, at block 840, the endoscope hookups on a third block of the universal connector are secured for fluid connection of the endoscope.

In some cases, the order of blocks 810 to 840 can be rearranged. For example, in some cases, the endoscope hookups on the third block of the universal connector can be secured first (block 840). In this case, the container with the endoscope can then be inserted into the reprocessing machine (block 810), followed by alignment (block 820 and block 830). In other examples, a different order of steps can be accomplished.

### VARIOUS NOTES & EXAMPLES

Example 1 is a universal connector assembly for securing a receptacle to a reprocessing system, the assembly comprising: a first block configured for aligning the receptacle to the reprocessing system in a first direction; a second block connectable to the first block, the second block configured for aligning the receptacle to the reprocessing system in a second direction; and a third block configured to receive one or more hook-ups.

In Example 2, the subject matter of Example 1 optionally includes a movement mechanism actuatable for connecting the third block to the first block through the second block.

In Example 3, the subject matter of Example 2 optionally includes wherein the movement mechanism comprises a manual handle.

In Example 4, the subject matter of any one or more of Examples 2-3 optionally include wherein the movement mechanism comprises an automated pulling actuator.

In Example 5, the subject matter of any one or more of Examples 1-4 optionally include wherein the first block comprises a fixed structure configured to receive one or more pins.

In Example 6, the subject matter of any one or more of Examples 1-5 optionally include wherein the second block comprises a spring-loaded attachment mechanism for securing the second block to the receptacle.

In Example 7, the subject matter of any one or more of Examples 1-6 optionally include wherein the third block is removable.

In Example 8, the subject matter of any one or more of Examples 1-7 optionally include wherein the third block comprises one or more endoscope hook-ups.

In Example 9, the subject matter of any one or more of Examples 1-8 optionally include wherein the first block is configured for vertical alignment of the receptacle.

In Example 10, the subject matter of any one or more of Examples 1-9 optionally include wherein the first block is configured for lateral alignment of the receptacle.

Example 11 is a reprocessing system comprising: a cleaning chamber; a tray separable from the system, the tray for insertion into the cleaning chamber; a universal connector configured to secure the tray to the reprocessing system in the cleaning chamber, the universal connector comprising: a first block configured for vertical connection of the tray to the reprocessing system; a second block connectable to the first block, the second block configured for lateral connection of the tray to the reprocessing system; a third block comprising one or more endoscope hook-ups; and a connecting portion actuatable for securing the second block to the third block and connecting the tray to the reprocessing system.

In Example 12, the subject matter of Example 11 optionally includes wherein the connecting portion comprises a handle.

In Example 13, the subject matter of any one or more of Examples 11-12 optionally include wherein the connecting portion comprises an automated trigger.

In Example 14, the subject matter of any one or more of Examples 11-13 optionally include wherein the one or more endoscope hook-ups comprises endoscope fluid flushing ports.

Example 15 is a method of connecting a container to a reprocessing system, the method comprising: vertically inserting the container into a cleaning chamber of the reprocessing system and vertically aligning the container to a first portion of a connector; laterally aligning the container within the cleaning chamber with a second portion of the connector; and hooking up one or more endoscope connectors to the container with a third portion of the connector.

In Example 16, the subject matter of Example 15 optionally includes securing the container to the cleaning chamber comprises manually moving a handle.

In Example 17, the subject matter of any one or more of Examples 15-16 optionally include securing the container to the cleaning chamber comprises automation.

Example 18 is a method of connecting an endoscope to an endoscope reprocessing system, the method comprising: laterally inserting a container into a cleaning chamber of the reprocessing system and vertically aligning the container to a first portion of a connector; vertically aligning the container within the cleaning chamber with a second portion of the connector; and hooking up one or more endoscope connectors to the container with a third portion of the connector.

In Example 19, the subject matter of Example 18 optionally includes securing the container to the cleaning chamber comprises manually moving a handle.

In Example 20, the subject matter of any one or more of Examples 18-19 optionally include securing the container to the cleaning chamber comprises automation.

Each of these non-limiting examples can stand on its own or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A universal connector assembly for securely connecting a receptacle to an endoscope reprocessing system, the assembly comprising:
a first block configured for aligning the receptacle to the endoscope reprocessing system in a first direction;
a second block connectable to the first block, the second block configured for aligning the receptacle to the endoscope reprocessing system in a second direction; and
a third block comprising one or more internal endoscope hook-ups, the third block configured to fit with the second block.

2. The assembly of claim 1, further comprising a movement mechanism actuatable for connecting the third block to the first block to the second block.

3. The assembly of claim 2, wherein the movement mechanism comprises a manual handle.

4. The assembly of claim 2, wherein the movement mechanism comprises an automated pulling actuator.

5. The assembly of claim 1, wherein the receptacle comprises a tray, rack, or container configured to receive an endoscope.

6. The assembly of claim 1, wherein the second block comprises a spring-loaded attachment mechanism for securing the second block to the receptacle.

7. The assembly of claim 1, wherein the third block is removable.

8. The assembly of claim 1, wherein the third block comprises one or more hook-ups configured to fluidly connect an endoscope thereto.

9. The connector of claim 1, wherein the first block is configured for vertical alignment of the receptacle.

10. The connector of claim 1, wherein the first block is configured for lateral alignment of the receptacle.

11. A reprocessing system comprising:
a cleaning chamber;
a tray separable from the system, the tray for insertion into the cleaning chamber; and
a universal connector configured to secure the tray to the reprocessing system in the cleaning chamber, the universal connector at least partially within a side of the tray, the universal connector comprising:
a first block configured for vertical connection of the tray to the reprocessing system;
a second block connectable to the first block, the second block configured for lateral connection of the tray to the reprocessing system;
a third block comprising one or more endoscope hook-ups; and
a connecting portion actuatable for securing the second block to the third block and connecting the connector to the reprocessing system.

12. The reprocessing system of claim 11, wherein the connecting portion comprises a handle.

13. The reprocessing system of claim 11, wherein the connecting portion comprises an automated trigger.

14. The reprocessing system of claim 11, wherein the one or more endoscope hook-ups comprises endoscope fluid flushing ports.

15. A method of connecting a receptacle to a reprocessing system, the method comprising:
vertically inserting the receptacle into a cleaning chamber of the reprocessing system and vertically aligning the receptacle to a first portion of a connector;
laterally aligning the receptacle within the cleaning chamber with a second portion of the connector; and
hooking up one or more endoscope connectors to the receptacle with a third portion of the connector.

16. The method of claim 15, further comprising securing the receptacle to the cleaning chamber by manually moving a handle to align the connector with the reprocessing system.

17. The method of claim 15, further comprising securing the receptacle to the cleaning chamber by invoking mechanical automation of the reprocessing system.

18. A method of connecting an endoscope to an endoscope reprocessing system, the method comprising:
laterally inserting a receptacle into a cleaning chamber of the reprocessing system and vertically aligning the receptacle to a first portion of a connector;
vertically aligning the receptacle within the cleaning chamber with a second portion of the connector; and
hooking up one or more endoscope connectors to the receptacle with a third portion of the connector.

19. The method of claim 18, further comprising securing the receptacle to the cleaning chamber by manually moving a handle.

20. The method of claim 18, further comprising securing the receptacle to the cleaning chamber by invoking mechanical automation of the reprocessing system.
